(19) United States
(12) Patent Application Publication
(10) Pub. No.: US 2013/0068457 A1
(43) Pub. Date: Mar. 21, 2013

(54) Method of Manufacture of Guerbet Alcohols For Making Surfactants Used In Petroleum Industry Operations

(71) Applicants: Sophany Thach, Houston, TX (US); Robert Shong, Houston, TX (US); Varadarajan Dwarakanath, Houston, TX (US); Gregory Winslow, Houston, TX (US)

(72) Inventors: Sophany Thach, Houston, TX (US); Robert Shong, Houston, TX (US); Varadarajan Dwarakanath, Houston, TX (US); Gregory Winslow, Houston, TX (US)

(73) Assignee: Chevron U.S.A. Inc., San Ramon, CA (US)

(21) Appl. No.: 13/233,406

(22) Filed: Sep. 15, 2011

(51) Int. Cl. C07C 303/24; C07C 303/28; E21B 43/22; C07C 41/03

(52) U.S. Cl. 166/270.1; 568/618; 558/41; 558/44; 558/34

(57) ABSTRACT

A method is disclosed for manufacturing surfactants for utilization in petroleum industry operations. The method comprises providing a bio-lipid. The bio-lipid can include one or more medium-chain or long-chain fatty acids, such as Lauric acid, Myristic acid, Palmitic acid, Stearic acid, Palmitoleic acid, Oleic acid, Ricinoleic acid, Vaccenic acid, Linoleic acid, Alpha-Linoleic acid, or Gamma-Linolenic acid. Fatty acid alkyl esters are produced by reacting the bio-lipid with a low-molecular weight alcohol. The fatty acid alkyl esters are reduced to a fatty alcohol. The fatty alcohol is dimerized to form a Guerbet alcohol, which is a precursor to producing surfactant for utilization in a petroleum industry operation, such as an enhanced oil recovery process.

### TECHNICAL FIELD

The present invention relates in general to the field of manufacturing Guerbet alcohols (GAs) for making surfactants, and more particularly, to a method of manufacturing large molecular-weight Guerbet alcohols from natural, hydrocarbon-independent sources of fat for making surfactants used in petroleum industry operations.

### BACKGROUND

Guerbet alcohols are a well-known type of alcohol formed via an aldol reaction. In particular, Guerbet alcohols are made through dimerization of an alcohol typically using catalysis. The produced Guerbet alcohols are beta-branched primary alcohols with twice the molecular weight of the reactant alcohols minus a mole of water. The overall reaction for preparing Guerbet alcohols can be represented by the following equation:

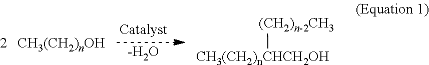

wherein subscript indice n is a positive integer greater or equal to 2. For example, if subscript indice n is eleven (11), the reactant alcohol has twelve carbon atoms (C₁₂) and the produced Guerbet alcohol has twenty-four carbon atoms (C₂₄). Similarly, C₁₆ alcohols (n=15) can be combined to make C₃₂ Guerbet alcohols. Examples of catalysts that can be used in preparing Guerbet alcohols include nickel, lead salts, oxides of copper, lead, zinc, chromium, molybdenum, tungsten, manganese, palladium compounds, silver compounds, or combinations thereof. Depending on the type of Guerbet alcohol to be produced, dimerization of the reactant alcohol can be carried out at temperatures ranging between about 100 to 300 Degrees Celsius.

The table below shows scientific names of C₆ to C₄₄ Guerbet alcohols and their corresponding chemical formulas.

| Guerbet Alcohol | Guerbet Formula |
|---|---|
| 2-Methyl-1-pentanol | C₆H₁₄O |
| 2-Ethyl-1-hexanol | C₈H₁₈O |
| 2-Propyl-1-heptanol | C₁₀H₂₂O |
| 2-Butyl-1-octanol | C₁₂H₂₆O |
| 2-Pentyl-1-nonanol | C₁₄H₃₀O |
| 2-Hexyl-1-decanol | C₁₆H₃₄O |
| 2-Heptyl-1-undecanol | C₁₈H₃₆O |
| 2-Octyl-1-dodecanol | C₂₀H₄₂O |
| 2-Nonyl-1-tridecanol | C₂₂H₄₆O |
| 2-Decyl-1-tetradecanol | C₂₄H₅₀O |
| 2-Undecyl-1-pentadecanol | C₂₆H₅₄O |
| 2-Dodecyl-1-hexadecanol | C₂₈H₅₈O |
| 2-Tridecyl-1-heptadecanol | C₃₀H₆₂O |
| 2-Tetradecyl-1-octadecanol | C₃₂H₆₆O |
| 2-Pentadecyl-1-nonadecanol | C₃₄H₇₀O |
| 2-Hexadecyl-1-eicosanol | C₃₆H₇₄O |
| 2-Heptadecyl-1-heneicosanol | C₃₈H₇₈O |
| 2-Octodecyl-1-docosanol | C₄₀H₈₂O |
| 2-Nonadecyl-1-tricosanol | C₄₂H₈₆O |
| 2-Eicosyl-1-tetraconsanol | C₄₄H₉₀O |

For most industrial applications, Guerbet alcohols are typically produced in high purity by driving the reaction (e.g., Equation 1) to near completion. Any unreacted monomer alcohol can be “stripped-off” to further enhance the purity of the produced Guerbet alcohol. As a result, highly branched, high molecular weight primary alcohols with near mid-point branching (i.e., large hydrophobes with high-purity beta branching) are produced. Guerbet alcohols tend to be more expensive than other alcohols due to the comprehensive conversion during the alcohol dimerization process and/or the subsequent removal of unreacted monomer alcohol. Accordingly, the cost of producing Guerbet alcohols can be prohibitive, especially for applications needing large quantities of Guerbet alcohols.

In petroleum industry applications, synthetic alcohols are currently the feedstock for synthesizing Guerbet alcohols. Synthetic alcohols are derived from hydrocarbon sources and therefore, the produced Guerbet alcohols further might not be economical if hydrocarbon prices are high. This is particularly the case as petroleum industry applications often require large quantities of Guerbet alcohols, such as for manufacturing surfactants.

### SUMMARY

A method is disclosed for manufacturing surfactants that are utilized in petroleum industry operations. The method includes providing a bio-lipid. The bio-lipid is reacted with low-molecular weight alcohol to produce fatty acid alkyl esters. For example, fatty acid alkyl esters can be produced by reacting triglycerides extracted from the bio-lipid with the low-molecular weight alcohol. The fatty acid alkyl esters are reduced to a fatty alcohol, such as by using a catalytic hydrogenation process. The fatty alcohol undergoes dimerization to form Guerbet alcohol. The Guerbet alcohol is used to produce surfactant, which is utilized in petroleum industry operations such as in an enhanced oil recovery process.

The bio-lipid can have a fatty acid composition that includes medium-chain fatty acids, long-chain fatty acids, or a combination thereof. In one or more embodiments, the bio-lipid includes one or more fatty acids having aliphatic tails of at least twelve carbon atoms. In one or more embodiments, the bio-lipid includes one or more fatty acids having aliphatic tails of at least sixteen carbon atoms. In one or more embodiments, the bio-lipid has a fatty acid composition of which at least fifty percent comprises Lauric acid, Myristic acid, Palmitic acid, Stearic acid, Palmitoleic acid, Oleic acid, Ricinoleic acid, Vaccenic acid, Linoleic acid, Alpha-Linoleic acid, Gamma-Linolenic acid, or a combination thereof.

In one or more embodiments, the fatty alcohol includes aliphatic alcohols having between twelve and eighteen carbon atoms. In one or more embodiments, the Guerbet alcohol includes beta-branched primary alcohols having between twenty-four and thirty-six carbon atoms.

In one or more embodiments, the surfactant is an alkoxylated Guerbet alcohol, which is formed by reacting lower weight epoxides with the Guerbet alcohol. In one or more embodiments, a Guerbet sulfate is formed by sulfation of the Guerbet alcohol. In one or more embodiments, a Guerbet sulfonate is formed by sulfonation of the Guerbet alcohol.

According to another aspect of the present invention, a method is disclosed for manufacturing surfactants that are utilized in petroleum industry operations. The method includes providing a blend of fatty acids including fatty acids extracted from a bio-lipid. For example, the blend of fatty acids can be in the form of triglycerides. The fatty acids extracted from the bio-lipid can include Lauric acid, Myristic acid, Palmitic acid, Stearic acid, Palmitoleic acid, Oleic acid, Ricinoleic acid, Vaccenic acid, Linoleic acid, Alpha-Linoleic acid, Gamma-Linolenic acid, or a combination thereof. The blend of fatty acids is reacted with low-molecular weight alcohol to produce fatty acid alkyl esters. The fatty acid alkyl esters are reduced to a fatty alcohol, such as by using a catalytic hydrogenation process. The fatty alcohol undergoes dimerization to form Guerbet alcohol. The Guerbet alcohol is used to produce surfactant, which is utilized in petroleum industry operations such as in an enhanced oil recovery process.

In one or more embodiments, the fatty acids extracted from the bio-lipid include one or more fatty acids having aliphatic tails of at least twelve carbon atoms. In one or more embodiments, the fatty acids extracted from the bio-lipid include one or more fatty acids having aliphatic tails of at least sixteen carbon atoms. In one or more embodiments, the fatty acids extracted from the bio-lipid have a fatty acid composition of which at least fifty percent comprises Lauric acid, Myristic acid, Palmitic acid, Stearic acid, Palmitoleic acid, Oleic acid, Ricinoleic acid, Vaccenic acid, Linoleic acid, Alpha-Linoleic acid, Gamma-Linolenic acid, or a combination thereof.

In one or more embodiments, the surfactant is an alkoxylated Guerbet alcohol, which is formed by reacting lower weight epoxides with the Guerbet alcohol. In one or more embodiments, a Guerbet sulfate is formed by sulfation of the Guerbet alcohol. In one or more embodiments, a Guerbet sulfonate is formed by sulfonation of the Guerbet alcohol.

According to another aspect of the present invention, a method is disclosed for enhancing hydrocarbon recovery in subterranean reservoirs. An injection well and a production well are provided that extend into and are in fluid communication with a hydrocarbon bearing zone of a subterranean reservoir. A solution, such as a sulfated alkoxylated Guerbet alcohol, is formed for injection into the hydrocarbon bearing zone from a Guerbet alcohol that is synthesized from a bio-lipid. The solution is injected into the hydrocarbon bearing zone of the reservoir and hydrocarbons from the hydrocarbon bearing zone of the subterranean reservoir are recovered through the production well.

In one or more embodiments, the Guerbet alcohol is synthesized from the bio-lipid. This is performed by extracting fatty acids from the bio-lipid, reacting the blend of fatty acids with a low-molecular weight alcohol to produce fatty acid alkyl esters, reducing the fatty acid alkyl esters to a fatty alcohol, and dimerizing the fatty alcohol to form the Guerbet alcohol.

The fatty acids extracted from the bio-lipid can include Lauric acid, Myristic acid, Palmitic acid, Stearic acid, Palmitoleic acid, Oleic acid, Ricinoleic acid, Vaccenic acid, Linoleic acid, Alpha-Linoleic acid, Gamma-Linolenic acid, or a combination thereof. In one or more embodiments, the fatty acids include one or more fatty acids having aliphatic tails of at least twelve carbon atoms. In one or more embodiments, the fatty acids include one or more fatty acids having aliphatic tails of at least sixteen carbon atoms. In one or more embodiments, the fatty acids have a fatty acid composition of which at least fifty percent comprises Lauric acid, Myristic acid, Palmitic acid, Stearic acid, Palmitoleic acid, Oleic acid, Ricinoleic acid, Vaccenic acid, Linoleic acid, Alpha-Linoleic acid, Gamma-Linolenic acid, or a combination thereof.

### BRIEF DESCRIPTION OF THE DRAWINGS

FIG. 1 is a schematic sectional view of a subterranean reservoir that is in fluid communication with an injection well and a production well, according to embodiments of the present invention.

### DETAILED DESCRIPTION

Aspects of the present invention describe a method for manufacturing large molecular-weight, highly branched Guerbet alcohols (GAs) from hydrocarbon-independent sources of fat. As will be described, the Guerbet alcohols according to embodiments of the present invention are produced from natural fats, which will be referred to herein as bio-lipids. This reduces the fluctuation and overall manufacturing costs of the Guerbet alcohols as bio-lipids do not depend on (i.e., are not synthesized from) hydrocarbon sources. Additionally, transportation costs can be reduced as bio-lipids can be grown in proximity (e.g., less than 100 miles) to where the products of Guerbet alcohols are utilized. 100181 The Guerbet alcohols manufactured from bio-lipids are precursors to surfactants. Guerbet alcohols possess many desirable physical properties for manufacturing surfactants. As previously discussed, Guerbet alcohols are high molecular weight primary alcohols with high-purity beta branching. Guerbet alcohols have low volatility and irritation properties compared to other linear alcohols. Melting point and viscosity are also reduced compared to other linear alcohols. They exhibit oxidative stability at high temperatures and remain liquid up until hydrocarbon chains lengths of C₂₀. Furthermore, Guerbet alcohols are reactive and can be used to make many derivatives, such as surfactants with a wide range of cloud points, which make them particularly suitable for many different petroleum industry operations. 100191 Surfactants can be utilized in various stages of hydrocarbon recovery and processing. Surfactants can be utilized in drilling operations (e.g., drilling fluids/dispersants), reservoir injection (e.g., fracturing fluids, enhanced oil recovery fluids), well productivity (e.g., acidizing fluids), hydrocarbon transportation, environmental remediation, or a combination thereof. Surfactants are commonly used when producing or transporting heavy or extra heavy oils, which generally have an API gravity of less than about 20 degrees API. As used herein, API gravity is the weight per unit volume of oil as measured by the American Petroleum Industries (API) scale. For example, API gravity can be measured according to the test methods provided by the American Society for Testing and Materials (ASTM) in test standard D287- 92(2006). Crude oil having an API gravity of less than about 20 degrees API is generally referred to as heavy oil. Crude oil having an API gravity of less than about 10 degrees API is generally referred to as extra heavy oil.

### Making of Natural Alcohols

Bio-lipids are used as the primary feedstock for Guerbet alcohols. Examples of bio-lipids include oleaginous plants or vegetables, animal fats, naturally-occurring triglycerides, genetically engineered triglycerides, or a combination thereof. This includes, but is not limited to, algal oil, canola oil, castor bean oil, coconut oil, corn oil, cotton oil, fish oil, flaxseed oil, hempseed oil, jatropha oil, lard, mustard seed oil, nut oil, olive oil, palm oil, palm kernel oil, peanut oil, rapeseed oil, safflower seed oil, soybean oil, sunflower oil, tall oil, tallow, yellow grease, or any oil produced by using bacteria (naturally-occurring or genetically engineered), yeast, fungi, unicellular organisms, and multicellular organisms. The table below shows chemical names and descriptions for common fatty acids found in the bio-lipids listed above. The number of double bonds between carbon atoms is also shown in the table below for the unsaturated fatty acids.

Examples of Fatty Acids

| | | | |
|---|---|---|---|
| | Chain | Double | |
| Fatty Acid | Length | Bonds | Scientific Name |

Saturated

| | | | |
|---|---|---|---|
| Butyric | C₄ | 0 | butanoic acid |
| Caproic | C₆ | 0 | hexanoic acid |
| Caprylic | C₈ | 0 | octanoic acid |
| Capric | C₁₀ | 0 | decanoic acid |
| Lauric | C₁₂ | 0 | dodecanoic acid |
| Myristic | C₁₄ | 0 | tetradecanoic acid |
| Palmitic | C₁₆ | 0 | hexadecanoic acid |
| Stearic | C₁₈ | 0 | octadecanoic acid |
| Arachidic | C₂₀ | 0 | eicosanoic acid |
| Behenic | C₂₂ | 0 | docosanoic acid |
| Lignoceric | C₂₄ | 0 | tetracosanoic acid |

Unsaturated

| | | | |
|---|---|---|---|
| Palmitoleic | C₁₆ | 1 | 9-hexadecenoic acid |
| Oleic | C₁₈ | 1 | 9-octadecenoic acid |
| Ricinoleic | C₁₈ | 1 | 12-hydroxy-9-octadecenoic acid |
| Vaccenic | C₁₈ | 1 | 11-octadecenoic acid |
| Linoleic | C₁₈ | 2 | 9,12-octadecadienoic acid |
| Alpha-Linolenic | C₁₈ | 3 | 9,12,15-octadecatrienoic acid |
| Gamma-Linolenic | C₁₈ | 3 | 6,9,12-octadecatrienoic acid |
| Gadoleic | C₂₀ | 1 | 9-eicosenoic acid |
| Arachidonic | C₂₀ | 4 | 5,8,11,14-eicosatetraenoic acid |
| EPA | C₂₀ | 5 | 5,8,11,14,17-eicosapentaenoic acid |
| Erucic | C₂₂ | 1 | 13-docosenoic acid |
| DHA | C₂₂ | 6 | 4,7,10,13,16,19-docosahexaenoic |
| | | | acid |

As used herein, the term “fatty acid” refers to a hydrocarbon chain having a terminal carboxyl group. In other words, a fatty acid is a carboxylic acid having an aliphatic tail (i.e., a straight or branched non-aromatic hydrocarbon chain). Fatty acids can be described according to the notation (x:y), where x represents the number of carbon atoms in the hydrocarbon chain and y represents the number of double bonds between carbon atoms. For example, C₁₆:2 represents 16 carbon atoms and 2 double bonds. Medium-chain fatty acids refer to fatty acids having aliphatic tails of six to twelve carbon atoms and long-chain fatty acids refer to fatty acids having aliphatic tails having greater than twelve carbon atoms. The fatty acids used herein are primarily extracted from the raw material of bio-lipids. Fatty acids often occur as triglycerides, which are three molecules of fatty acids (same or different, e.g., two radicals of oleic acid and one of palmitic acid) joined at the carboxyl groups via ester bonds to hydroxyl groups of glycerol. The fatty acids of the triglyceride can be saturated, monounsaturated, or polyunsaturated.

In one embodiment, coconut oil is extracted from the kernels or meat of coconut fruit harvested from coconut trees. Coconut oil is mainly comprised of glycerol esters of medium-chain and long-chain fatty acids, about half (e.g., 45-55%) of which is Lauric acid (saturated C₁₂ fatty acid). Coconut oil also typically comprises about eight to about twenty percent each of Myristic acid (saturated C₁₄ fatty acid) and Palmitic acid (saturated C₁₆ fatty acid), and comprises about five to about ten percent each of Caprylic acid (saturated C₈ fatty acid), Capric acid (saturated C₁₀ fatty acid), and Oleic acid (monounsaturated C₁₈ fatty acid). As will be described later herein, because coconut oil is rich in C₁₂ fatty acids, it is a particularly suitable feedstock for C₂₄ Guerbet alcohol. Further, currently many of the world's largest producers of coconut oil (e.g., Southeast Asia) are within close proximity to petroleum industry operations where the products of Guerbet alcohols, such as surfactants, can be utilized.

In one embodiment, palm oil is extracted from the pulp of fruit harvested from palm trees. Palm oil is mainly comprised of glycerol esters of long-chain fatty acids, about forty to about fifty percent of which is Palmitic acid (saturated C₁₆ fatty acid). Palm oil also typically comprises about thirty to about forty percent of Oleic acid (monounsaturated C₁₈ fatty acid), about five to about fifteen percent of Linoleic acid (polyunsaturated C₁₈ fatty acid), and about three to six percent of Stearic acid (saturated C₁₈ fatty acid). As will be described later herein, because palm oil is rich in C₁₆ and C₁₈ fatty acids, it is a particularly suitable feedstock for C₃₂ and C₃₆ Guerbet alcohols. Further, currently many of the world's largest producers of palm oil (e.g., Southeast Asia) are within close proximity to petroleum industry operations where the products of Guerbet alcohols can be utilized.

In one embodiment, palm kernel oil is extracted from the kernels of palm trees. Palm kernel oil is mainly comprised of glycerol esters of medium-chain and long-chain fatty acids, about forty-five to about fifty-five percent of which is Lauric acid (saturated C₁₂ fatty acid). Palm kernel oil also typically comprises about fifteen to about twenty percent each of Myristic acid (saturated C₁₄ fatty acid) and Oleic acid (monounsaturated C₁₈ fatty acid), and comprises about five to about ten percent of Palmitic acid (saturated C₁₆ fatty acid). As will be described later herein, because palm kernel oil is rich in C₁₂ fatty acids, it is a particularly suitable feedstock for C₂₄ Guerbet alcohol. Similar to palm oil, the world's largest producers of palm kernel oil are within close proximity to petroleum industry operations where the products of Guerbet alcohols can be utilized.

In one embodiment, oil is extracted from the castor bean. Castor bean oil is mainly comprised of glycerol esters of long-chain fatty acids, about eight-five percent to about ninety-five percent of which is Ricinoleic acid (monounsaturated C₁₈ fatty acid). It also typically comprises about one to six percent each of Oleic acid (monounsaturated C₁₈ fatty acid) and Linoleic acid (polyunsaturated C₁₈ fatty acid). As will be described later herein, because castor bean oil is rich in C₁₈ fatty acids, it is a particularly suitable feedstock for C₃₆ Guerbet alcohol. India, Brazil, and China are currently the largest producers of castor bean oil.

In one embodiment, nut oil is utilized. For example, the nut oils can be comprised of glycerol esters of medium-chain and long-chain fatty acids. For example, some nut oils comprise about thirty-five to about sixty percent of Oleic acid (monounsaturated C₁₈ fatty acid) and about ten to about forty percent of Linoleic acid (polyunsaturated C₁₈ fatty acid). Nut oils can also comprise about five to about fifteen percent of Palmitic acid (saturated C₁₆ fatty acid) and about two to six percent of Stearic acid (saturated C₁₈ fatty acid). As will be described later herein, because nut oil is rich in C₁₆ and C₁₈ fatty acids, it is a particularly suitable feedstock for C₃₂ and C₃₆ Guerbet alcohols.

In one embodiment, a blend of medium-chain and/or long-chain fatty acids is utilized for manufacturing Guerbet alcohols. The blend of fatty acids can be fully or partially extracted from one or more bio-lipids. The blend can include a high percentage of C₁₂ through C₁₈ fatty acids, such as Lauric acid (C₁₂:0), Myristic acid (C₁₄:0), Palmitic acid (C₁₆:0), Stearic acid (C₁₈:0), Palmitoleic acid (C₁₆:1), Oleic acid (C₁₈:1), Ricinoleic acid (C₁₈:1), Vaccenic acid (C₁₈:1), Alpha-Linoleic acid (C₁₈:2), Gamma-Linolenic acid (C₁₈:3), or a combination thereof. For example, the percentage of C₁₂ through C₁₈ fatty acids in the blend can be greater than about 50 percent. In another example, the percentage of C₁₂ through C₁₈ fatty acids in the blend is greater than about 80 percent. In another example, the percentage of C₁₂ through C₁₈ fatty acids in the blend is greater than about 90 percent. In each of these embodiments, while a fatty acid composition might be rich in C₁₂ through C,₈ fatty acids, it can also contain fatty acids smaller than C₁₂ and greater than C₁₈, such as C₈ or C₂₀ fatty acids, respectively. Medium-chain and long-chain fatty acids are particularly.useful for making highly branched, high molecular weight primary alcohols (Guerbet alcohols), which can then be used for making very large hydrophobe surfactants that are used for obtaining ultra-low interfacial tensions and low micro-emulsion viscosities.

Other fatty acid compositions of bio-lipids can be found in the following publications:
Swern, D., “Bailey's Industrial Oil and Fat Products,” 3^{rd} ed., Interscience Publishers, New York, N.Y., 1964, pp. 176 and 192.
Ang, Catharina Y. W., KeShun Liu, and Yao-Wen Huang, “Asian Foods: Science and Technology,” Technomic Publishing Company, Inc., Lancaster, Pa., 1999.
Fife, Bruce, “Coconut Cures,” Piccadilly Books, Ltd., Colorado Springs, Colo., 2005, pp. 184 and 185.
Knothe, G., Dunn, R. O., Bagby, M. O., “Biodiesel: The Use of Vegetable Oils and Their Derivatives as Alternative Diesel Fuels.” Fuels and Chemicals from Biomass. Presented at American Chemical Society Symposium, Ser. 666, Washington D.C., 1997.

There are many processes for breaking down the triglyceride bonds to convert the aforementioned bio-lipids to fatty acid alkyl esters such as transesterification, blending, microemulsions, and pyrolysis. Transesterification is the most common method used for producing fatty acid alkyl esters from bio-lipid. The term “transesterification” (as well as derivatives, other forms of this term, and linguistically related words and phrases), as used herein, generally refers to the process of forming an ester by reacting one or more fatty acids with an alcohol, typically in the presence of a catalyst. More specifically, this term refers to the process of converting bio-lipids to fatty acid alkyl esters and glycerin. Generally, the bio-lipid raw materials, or the fatty acids and triglycerides obtained after subjecting the bio-lipid raw materials to separation, are reacted with a low-molecular weight alcohol in the presence of a catalyst to produce fatty acid alkyl esters and glycerin. In most applications, the low-molecular weight alcohol is methanol or ethanol. Other possible low-molecular weight alcohols include propanol and butanol. Catalysts accelerate the chemical reaction by reducing the activation energy (i.e., the energy needed to initiate the reaction). Examples of catalysts (or biocatalysts) include acids (e.g., hydrochloric acid, sulfuric acid, sulfonic acid, heteropoly acid, a Lewis acid, a Bronsted acid), a Bronsted acidic ionic liquid, organic or inorganic bases, enzymes, lipase, and an alkoxide, a carbonate, or a hydroxide of sodium, potassium, calcium, or barium. Sodium hydroxide (NaOH), potassium hydroxide (KOH), sodium methoxide (NaOCH₃), and potassium methoxide (KOCH₃) are the most common alkali catalysts used for transesterification.

For example, the transesterification of a triglyceride with methanol to produce methyl ester and glycerin is represented by the following equation, where sodium methoxide is used as a used as the base catalyst:

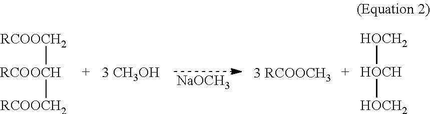

In Equation 2, R represents an aliphatic group, such as an alkyl group, comprising typically between about 4 and about 22 carbon atoms. The triglycerides react with the low molecular weight alcohol to convert molecules of fat to fatty acid alkyl esters and glycerin. The fatty acid alkyl esters can be separated from the glycerin during the transesterification reaction or after its completion. For example, separation can be accomplished using a separator, a centrifuge, a filtration mechanism, adsorption, distillation, extraction, suitable reagents, or by allowing the glycerin to naturally settle due to gravity.

Accordingly, transesterification of the bio-lipid results in one or more fatty acid alkyl esters including, but not limited to, algal oil alkyl ester, castor bean oil alkyl ester, canola oil alkyl ester, coconut oil alkyl ester, corn oil alkyl ester, cotton oil alkyl ester, fish oil alkyl ester, flaxseed oil alkyl ester, hempseed oil alkyl ester, jatropha oil alkyl ester, lard alkyl ester, mustard seed oil alkyl ester, nut oil alkyl ester, olive oil alkyl ester, palm oil alkyl ester, palm kernel oil alkyl ester, peanut oil alkyl ester, rapeseed oil alkyl ester, safflower seed oil alkyl ester, soybean oil alkyl ester, sunflower oil alkyl ester, tall oil alkyl ester, tallow alkyl ester, yellow grease alkyl ester, or any alkyl ester produced from an oil of a bacteria (naturally-occurring or genetically engineered), yeast, fungi, unicellular organism, or multicellular organism.

The fatty acid alkyl esters, such as fatty acid methyl ester, are then reduced to fatty alcohols (natural alcohols), which typically are aliphatic alcohols having a chain of 8 to 22 carbon atoms. In one embodiment, the esters of fatty acids are hydrogenated using a catalyst, such as copper chromite. For example, the catalytic hydrogenation of fatty acid methyl ester producing a fatty alcohol and methanol is represented by the following equation:

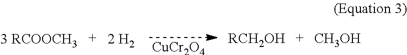

wherein R represents an aliphatic group (either a straight or branched non-aromatic hydrocarbon chain), such as an alkyl group. Functional group R typically comprises between about 4 and about 22 carbon atoms.

As previously described, the fatty or natural alcohols can be used to produce Guerbet alcohols through a dimerization process. Accordingly, the Guerbet alcohols described herein are produced from hydrocarbon-independent sources of fat (i.e., natural fats or bio-lipids). This reduces the fluctuation and overall manufacturing costs of the Guerbet alcohols as the bio-lipids are not synthesized from hydrocarbon sources. Furthermore, the bio-lipids can be grown in proximity to where the products of the Guerbet alcohols are utilized, which can reduce transportation costs. For example, the Guerbet alcohols can be used in hydrocarbon recovery and processing. In one embodiment, the bio-lipids are grown within about 500 miles of a reservoir field that produces hydrocarbons from a subterranean reservoir. In one embodiment, the bio-lipids are grown within about 100 miles of a reservoir field that produces hydrocarbons from a subterranean reservoir. In one embodiment, the bio-lipids are grown within about 50 miles of a reservoir field that produces hydrocarbons from a subterranean reservoir.

In one embodiment, the Guerbet alcohols are utilized to manufacture surfactants, which, for example, can be used as wetting agents, emulsifiers, detergents and solubilizers. As previously discussed, Guerbet alcohols have many physical properties that make them beneficial for making very large hydrophobe surfactants, which can be used to obtain ultra-low interfacial tensions and low micro-emulsion viscosities. Surfactants are commonly used in the petroleum industry for drilling operations (e.g., drilling fluids/dispersants), reservoir injection (e.g., fracturing fluids, enhanced oil recovery fluids), well productivity (e.g., acidizing fluids), hydrocarbon transportation, environmental remediation, or a combination thereof. The selection of a surfactant for a petroleum industry application typically depends on various factors such as total acid number (TAN), crude-oil composition in the reservoir, and the compatibility with the make-up or injection brine. Standard phase-behavior tests can be conducted to screen for appropriate surfactants.

In some embodiments, Guerbet alcohols are alkoxylated to form alkoxylated Guerbet alcohols. Here, lower weight epoxides, such as ethylene oxide (EO), propylene oxide (PO) and butylene oxide (BO), are added to the Guerbet alcohols. In some embodiments, more than six (6) repeating units, such as EO, are present. In some embodiments, more than ten to twenty repeating units, such as EO, are present. These lower weight epoxides are typically used to tailor the surfactant such that it exhibits a desirable phase behavior for particular reservoir conditions, such as electrolyte concentrations (salinities), temperature, and hydrocarbon compositions. Accordingly, a desired HLB (Hydrophillic-Lipophillic-Balance) can be achieved by tailoring the number of alkoxylates attached to the Guerbet alcohol.

In some embodiments, Guerbet alcohols are sulfated to obtain Guerbet sulfates. For example, sulfamic acid sulfation can be used. In some embodiments, Guerbet alcohols are sulfonated to obtain Guerbet sulfonates. Alkoxylated Guerbet alcohols can also undergo sulfation or sulfonation to produce large, branched C₂₄ - C₃₂ alkyl alkoxylated surfactants, such as alkyl sulfate surfactants or alkyl sulfonate surfactants. These surfactants can also be tailored to exhibit desirable phase behaviors for particular reservoir conditions by altering the molecular weight, molecular weight distribution, and branching/point of attachment (e.g., attachment of aryl groups to alkyl groups).

One example of a surfactant that can be manufactured from a Guerbet alcohol is an anionic surfactant. Anionic surfactants, such as sulfates, sulfonates, phosphates, and carboxylates are known and described in the art in, for example, SPE 129907 and U.S. Pat. No. 7,770,641, which are both incorporated herein by reference. Non-ionic surfactants can also be manufactured from Guerbet alcohols. Examples of non-ionic surfactants include alcohol alkoxylates such as alkylaryl alkoxy alcohols or alkyl alkoxy alcohols. Currently available alkoxylated alcohols include Lutensol® TDA 10EO and Lutensol® OP40, which are manufactured by BASF SE headquartered in Rhineland-Palatinate, Germany. Neodol 25, which is manufactured by Shell Chemical Company, is also a currently available alkoxylated alcohol. Chevron Oronite Company LLC, a subsidiary of Chevron Corporation, also manufactures alkoxylated alcohols such as L24-12 and L14-12, which are twelve-mole ethoxylates of linear carbon chain alcohols. In some embodiments, non-ionic surfactants manufactured from Guerbet alcohols are combined with other non-ionic surfactants such as non-ionic esters.

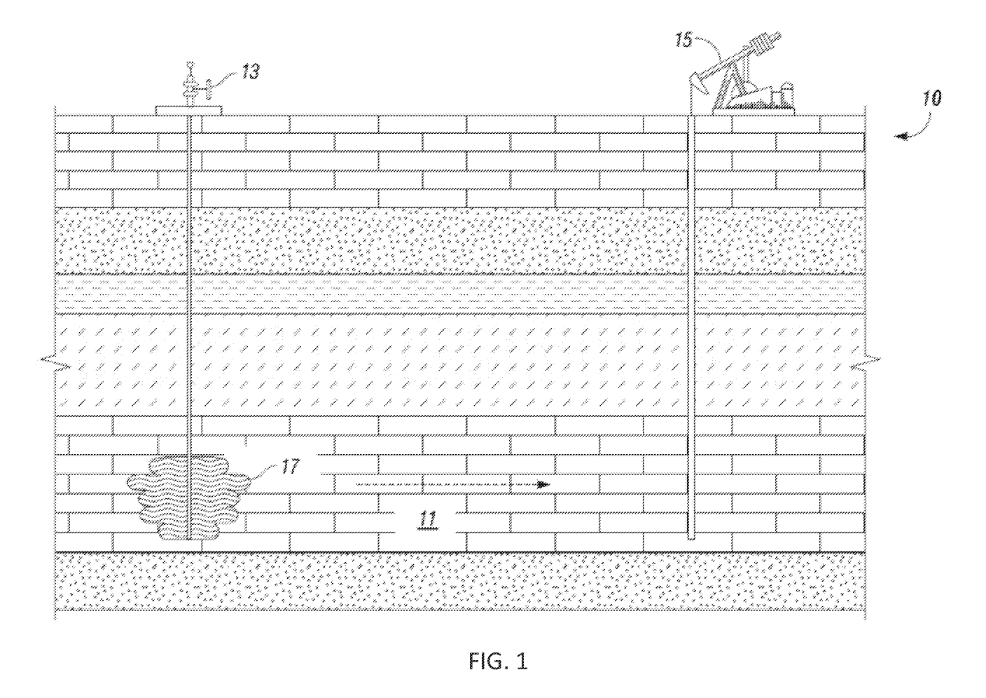

Referring to FIG. 1, a cross-section of subterranean reservoir 10 is shown. Subterranean reservoir 10 includes a plurality of rock layers including hydrocarbon bearing strata or zone 11. Subterranean reservoir 10 can be any type of subsurface formation in which hydrocarbons are stored, such as limestone, dolomite, oil shale, sandstone, or a combination thereof. Injection well 13 extends into hydrocarbon bearing zone 11 of subterranean reservoir 10 such that injection well 13 is in fluid communication with hydrocarbon bearing zone 11. Production well 15 is also in fluid communication with hydrocarbon bearing zone 11 of subterranean reservoir 10 in order to receive hydrocarbons therefrom. Production well 15 is positioned a predetermined lateral distance away from injection well 13. For example, production well 15 can be positioned between 100 feet to 10,000 feet away from injection well 13. As will be readily appreciated by those skilled in the art, additional injection wells 13 and production wells 15 can extend into subterranean reservoir 10 such that multiple production wells 15 optimally receive hydrocarbons being pushed through hydrocarbon bearing zone 11 due to injections from multiple injection wells 13. Furthermore, while not shown in FIG. 1, injection well 13 and production well 15 can deviate from t that in some embodiments, injection well 13 and/or production well 15 can be a directional well, horizontal well, or a multilateral well.

In one embodiment, a solution 17 is injected into hydrocarbon bearing zone 11 of subterranean reservoir 10 through injection well 13. Solution 17 comprises a chemical composition, such as a surfactant, manufactured from Guerbet alcohols synthesized from one or more hydrocarbon-independent sources of fat (i.e., natural fats or bio-lipids). As previously described, Guerbet alcohols can be synthesized from a bio-lipid by extracting the blend of fatty acids (e.g., Lauric acid, Myristic acid, Palmitic acid, Stearic acid, Palmitoleic acid, Oleic acid, Ricinoleic acid, Vaccenic acid, Linoleic acid, Alpha-Linoleic acid, and Gamma-Linolenic acid) contained therein. Fatty acid alkyl esters are then produced by reacting the blend of fatty acids with a low-molecular weight alcohol. The fatty acid alkyl esters are reduced to a fatty alcohol, which is then dimerized to form the Guerbet alcohol. If solution 17 is a surfactant, the Guerbet alcohol can be reacted with lower weight epoxides to form an alkoxylated Guerbet alcohol, which can further be sulfated or sulfonated. Such surfactants can penetrate into pore spaces of the reservoir formation's rock matrix contacting trapped oil globules, thereby reducing the interfacial tension between the water and oil in the reservoir and releasing the oil from the pore spaces. Surfactants can be injected in any manner such as in an aqueous solution, a surfactant-polymer (SP) flood or an alkaline-surfactant-polymer (ASP) flood. The surfactants can be injected continuously or in a batch process.

In one embodiment, surfactants are utilized for environmental treatment of wastes (ex situ and/or in situ). In particular, at least one surfactant manufactured using Guerbet alcohols synthesized from one or more hydrocarbon-independent sources of fat is used to enhance chemical treatment of contaminated soil or sediment. The contaminant may be organic, such as oil or solvent, or inorganic, such as mercury and arsenic. The surfactant reduces the icial tension between oil and water, thereby increasing the solubility of the contaminant.

While in the foregoing specification this invention has been described in relation to certain preferred embodiments thereof, and many details have been set forth for purpose of illustration, it will be apparent to those skilled in the art that the invention is susceptible to alteration and that certain other details described herein can vary considerably without departing from the basic principles of the invention. For example, the product of the Guerbet alcohol synthesized from the bio-lipid can be mixed with additional chemicals (e.g., solvents, co-solvents, co-surfactants, polymers) prior to use in petroleum industry applications.

What is claimed is:

1. A method for manufacturing surfactants that are utilized in petroleum industry operations, the method comprising:
(a) providing a bio-lipid;
(b) reacting the bio-lipid with a low-molecular weight alcohol to produce fatty acid alkyl esters;
(c) reducing the fatty acid alkyl esters to a fatty alcohol;
(d) dimerizing the fatty alcohol to form a Guerbet alcohol;
(e) producing a surfactant from the Guerbet alcohol; and
(f) utilizing the surfactant in a petroleum industry operation.

2. The method of claim 1, wherein the bio-lipid has a fatty acid composition including at least one medium-chain fatty acid.

3. The method of claim 1, wherein the bio-lipid has a fatty acid composition including at least one long-chain fatty acid.

4. The method of claim 1, wherein the bio-lipid has a fatty acid composition including one or more fatty acids having aliphatic tails of at least twelve carbon atoms.

5. The method of claim 1, wherein the bio-lipid has a fatty acid composition including one or more fatty acids having aliphatic tails of at least sixteen carbon atoms.

6. The method of claim 1, wherein the bio-lipid has a fatty acid composition of which at least fifty percent comprises Laurie acid, Myristic acid, Palmitic acid, Stearic acid, Palmitoleic acid, Oleic acid, Ricinoleic acid, Vaccenic acid, Linoleic acid, Alpha-Linoleic acid, Gamma-Linolenic acid, or a combination thereof

7. The method of claim 1, wherein the fatty acid alkyl esters are produced in step (b) by reacting triglycerides extracted from the bio-lipid with the low-molecular weight alcohol.

8. The method of claim 1, wherein the fatty acid alkyl esters are reduced to the fatty alcohol in step (c) using a catalytic hydrogenation process.

9. The method of claim 1, wherein the fatty alcohol includes aliphatic alcohols having between twelve and eighteen carbon atoms.

10. The method of claim 1, wherein the Guerbet alcohol includes beta-branched primary alcohols having between twenty-four and thirty-six carbon atoms.

11. The method of claim 1, wherein the producing the surfactant from the Guerbet alcohol in step (e) comprises forming an alkoxylated Guerbet alcohol by reacting lower weight epoxides with the Guerbet alcohol.

12. The method of claim 1, wherein the producing the surfactant from the Guerbet alcohol in step (e) comprises forming a Guerbet sulfate by sulfation of the Guerbet alcohol.

13. The method of claim 1, wherein the producing the surfactant from the Guerbet alcohol in step (e) comprises forming a Guerbet sulfonate by sulfonation of the Guerbet alcohol.

14. The method of claim 1, wherein utilizing the surfactant in a petroleum industry operation comprises injecting the surfactant into a subterranean reservoir in an enhanced oil recovery process.

15. A method for manufacturing surfactants that are utilized in petroleum industry operations, the method comprising:
(a) providing a blend of fatty acids including fatty acids extracted from a bio-lipid, the fatty acids extracted from the bio-lipid including at least one fatty acid selected from the group consisting of Laurie acid, Myristic acid, Palmitic acid, Stearic acid, Palmitoleic acid, Oleic acid, Ricinoleic acid, Vaccenic acid, Linoleic acid, Alpha-Linoleic acid, and Gamma-Linolenic acid;
(b) reacting the blend of fatty acids with a low-molecular weight alcohol to produce fatty acid alkyl esters;
(c) reducing the fatty acid alkyl esters to a fatty alcohol;
(d) dimerizing the fatty alcohol to form a Guerbet alcohol;
(e) producing a surfactant from the Guerbet alcohol; and
(f) utilizing the surfactant in a petroleum industry operation.

16. The method of claim 15, wherein the fatty acids extracted from the bio-lipid comprise triglycerides.

17. The method of claim 15, wherein the producing the surfactant from the Guerbet alcohol comprises:
(i) reacting the Guerbet alcohol with lower weight epoxides to form an alkoxylated Guerbet alcohol; and
(ii) sulfating the alkoxylated Guerbet alcohol.

18. A method for enhancing hydrocarbon recovery in subterranean reservoirs, the method comprising:
(a) providing an injection well and a production well that extend into a hydrocarbon bearing zone of a subterranean reservoir and are in fluid communication therewith;
(b) forming a solution for injection into the hydrocarbon bearing zone from a Guerbet alcohol that is synthesized from a bio-lipid;
(c) injecting the solution into the hydrocarbon bearing zone of the reservoir; and
(d) recovering hydrocarbons from the hydrocarbon bearing zone of the subterranean reservoir through the production well.

19. The method of claim 18, wherein the Guerbet alcohol is synthesized from the bio-lipid by:
(i) extracting fatty acids from the bio-lipid, the fatty acids extracted from the bio-lipid including at least one fatty acid selected from the group consisting of Lauric acid, Myristic acid, Palmitic acid, Stearic acid, Palmitoleic acid, Oleic acid, Ricinoleic acid, Vaccenic acid, Linoleic acid, Alpha-Linoleic acid, and Gamma-Linolenic acid;
(ii) reacting the blend of fatty acids with a low-molecular weight alcohol to produce fatty acid alkyl esters;
(iii) reducing the fatty acid alkyl esters to a fatty alcohol; and
(iv) dimerizing the fatty alcohol to form the Guerbet alcohol.

20. The method of claim 18, wherein the forming the solution for injection into the hydrocarbon bearing zone from the Guerbet alcohol in step (b) further comprises:
(i) reacting the Guerbet alcohol with lower weight epoxides to form an alkoxylated Guerbet alcohol; and
(ii) sulfating the alkoxylated Guerbet alcohol.
